(19) United States
(12) Patent Application Publication
(10) Pub. No.: US 2004/0242944 A1
(43) Pub. Date: Dec. 2, 2004

(54) Method for the production of propene by metathesis of c4 to c9-olefins

(76) Inventors: Michael Röper, (US); Jürgen Stephan, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/488,272

(22) PCT Filed: Aug. 28, 2002
(86) PCT No.: PCT/EP02/09580

(30) Foreign Application Priority Data
Sep. 4, 2001 (DE) 101 43 160.0

(51) Int. Cl. C07C 2/00

(52) U.S. Cl. 585/324

(57) ABSTRACT

The invention relates to a method for the production of propene by multiple execution of the following sequence of steps, whereby in step 1 a mixture M1, essentially comprising unbranched C₄- to C₉ monoolefins (Components 04-9), 0 to 15 mol. % ethylene (component E), based on the components 04-9, 0 to 100 mol. % saturated hydrocarbons (components KG), based on the components 04-9, 0 to 30 mol. % olefins with more than 9 hydrocarbon atoms (components 0>9), based on components 04-9, 0 to 1 mol. %, based on the components 04-9, propene (component 03), 0 to 10 mol. %, based on the components 04-9, of other hydrocarbons (components KS), is brought into contact with a metathesis catalyst at a temperature of 20 to 350° C., with the proviso that the molar proportion of the sum of the 1-olefins and the sum of the 2-olefins to the sum of the components 04-9 and 0>9 is at least 0.5%, so long as the mixture M1 is not simultaneously or previously subjected to an isomerisation, correspondingly changing the proportion of 1- and 2-olefins, thus producing a mixture M2. In step II a mixture M1 is produced from a mixture M2 whereby a) propene and optionally components 0>9 and components KS are completely or partially removed and b) amounts of components 04-09 are added, a maximum of 1 mol. % of component E, based on the added amounts of components 04-09 and optionally other saturated or olefinically-unsaturated hydrocarbons, based on the added amounts of components 04-09, are added.

The present invention relates to a process for preparing propene by carrying out the following sequence of steps a plurality of times, wherein

I. in step I, a mixture M1 consisting essentially of

unbranched C₄-C₉-monoolefins (components O4-9),

from 0 to 15 mol % of ethylene (component E), based on the components O4-9,

from 0 to 100 mol % of saturated hydrocarbons (components HS), based on the components O4-9,

from 0 to 30 mol %, based on the components O4-9, of olefins having more than 9 carbon atoms (components O>9)

from 0 to 1 mol %, based on the components O4-9, of propene (component O3)

from 0 to 10 mol %, based on the components O4-9, of other hydrocarbons (components HO)

is brought into contact with a metathesis catalyst at from 20 to 350° C., with the proviso that the mole fraction of all 1-olefins and of all 2-olefins as a proportion of the total components O4-9 and O>9 is in each case at least 0.5% if the mixture M1 is not subjected simultaneously or beforehand to an isomerization by means of which the proportion of 1- and 2-olefin is set accordingly, and a mixture M2 is thus prepared,

II. in step II, a mixture M1 is prepared from the mixture M2 by

a) removing propene and, if appropriate, components O>9 and components HO either completely or partly and

b) adding components O4-O9, a maximum of 1 mol % of component E, based on the amounts of components O4-O9 added, and, if appropriate, other saturated or olefinically unsaturated hydrocarbons.

It is generally known that steam crackers operated using naphtha are employed, in particular, for providing unsaturated hydrocarbons which can serve as starting materials for the preparation of further higher value added organic compounds. Particularly valuable starting materials are ethylene, propylene, butenes and hydrocarbons containing a phenyl group. Since, on the one hand, the product spectrum of a steam cracker can only be influenced within narrow limits as far as the abovementioned desired products are concerned but, on the other hand, the demand for the individual products is sometimes very different, there is particular interest in providing processes which can interconvert these products, some of which may be needed to a lesser extent depending on locality or time, so as to be able to react flexibly to the current demand for the individual products of value.

A frequently encountered problem is that olefins having 4 or more carbon atoms are available in ample quantities, but propene and ethylene are particularly sought after.

Processes for preparing propene from other olefinic hydrocarbons by means of metathesis reactions are known from, for example, the following documents.

U.S. Pat. No. 3,785,957 discloses the preparation of propene by reaction of 1-butene with 2-butene having a high ethylene content over MoO₃ and CoO on Al₂O₃.

EP-A-691318 relates to a process for preparing butenes and propene from ethylene and pentenes.

DE-A-19932060 discloses a process for preparing C5-/C6-olefins, with propene likewise being produced. Here, butenes are subjected to a metathesis and ethylene, propene, pentenes and hexenes are separated off from the reaction mixture. Only the unreacted butene is recirculated as a recycle stream.

Both DE-A-19813720 and DE-A-10013253, 10130958 and 10118634 which are not prior publications relate to processes for converting C₄ and higher olefins into propene. However, the processes employ relatively large amounts of ethylene which is consumed in the metathesis.

It is an object of the present invention to provide a process by means of which propene can be prepared in a targeted manner from essentially only olefinic hydrocarbons having 4 and more carbon atoms.

The mixture M1 used according to the present invention preferably consists essentially of

unbranched C₄-C₉-monoolefins (components O4-9),

from 0.01 to 15 mol % of ethylene (component E), based on the components O4-9,

from 0 to 100 mol % of saturated hydrocarbons (components HS), based on the components O4-9,

from 0 to 30 mol %, based on the components O4-9, of olefins having more than 9 carbon atoms (components O>9)

from 0 to 1 mol %, based on the components O4-9, of propene (component O3)

from 0 to 10 mol %, based on the components O4-9, of other hydrocarbons (components HO).

Suitable components O4-9 are, in particular, 1- and 2-butene, n-pentenes, n-hexenes, n-octenes and n-nonenes.

The components O>9 are, in particular, unbranched hydrocarbons having one olefinic double bond, e.g. decenes, undecenes or dodecenes and homologues.

Possible saturated hydrocarbons (components HS) are, in particular, saturated hydrocarbons such as ethane, propane, butane, isobutane, neopentane, isopentane, methylcyclopropane. Possible other hydrocarbons (components HO) are, in particular, aromatic hydrocarbons such as benzene or styrene or multiply unsaturated hydrocarbons such as 1,3-butadiene.

The amounts of 1- and 2-olefins as a proportion of the total components O4-9 and O>9 can be chosen freely if the metathesis reaction is carried out under conditions under which a random isomerization of components O4-9 takes place at the same time. If this is not the case, the mole fraction of all 1-olefins and of all 2-olefins as a proportion of the total components O4-9 and O>9 is in each case at least 0.5%.

As metathesis catalysts with which the mixture M1 is brought into contact for the desired reaction when the proportion of 1- and 2-isomers is in accordance with the definition, it is possible to use catalysts comprising a compound of a metal of group VIb or VIIb of the Periodic Table of the Elements. The metathesis catalyst preferably comprises an oxide of a metal of group VIb or VIIb of the Periodic Table. In particular, the metathesis catalyst is selected from the group consisting of Re₂O₇, WO₃ and MoO₃. Such suitable catalysts and their preparation are described, for example, in DE-A-10013253.

The reaction can be carried out either in the liquid phase or in the gas phase.

In the liquid phase, the metathesis is preferably carried out at from 0 to 110° C., while it is preferably carried out at from 150 to 350° C. in the gas phase.

The pressure is generally from 10 to 15 bar if the reaction is carried out in the liquid phase and from 1 to 5 bar if it is carried out in the gas phase.

Reaction times of from 1 to 5 hours are usually sufficient.

If the metathesis is to be carried out under isomerizing conditions, which is generally preferred regardless of the proportion of 1- and 2-olefins based on the components O4-9 and O>9 in the mixture M1, 2 possibilities are available:

1. The metathesis is carried out at from 110 to 350° C. and pressures of from 1 to 60 bar, particularly preferably at about 150° C. and about 5 bar, using Re₂O₇ on Al₂O₃ as catalyst.

2. Catalyst packing comprising not only the abovementioned metathesis catalysts but also isomerization catalysts different therefrom is used. The isomerization catalysts comprise a metal of group Ia, IIa, IIIb, IVb, Vb or VIII of the Periodic Table of the Elements or a compound thereof. The isomerization catalyst is preferably selected from the group consisting of RuO₂, MgO and K₂CO₃.

The catalysts are generally supported on the customary materials known to those skilled in the art. Examples of suitable materials include SiO₂, gamma-Al₂O₃, MgO or mixtures of these materials.

The reaction of the present invention can be carried out batchwise or continuously, for example by continuously producing a liquid or gaseous stream formed by the mixture M1 into a reaction zone, bringing it into contact with the metathesis catalyst there and continuously taking a stream II from the reaction zone.

In step II, a mixture M1 is generally once again prepared continuously from the mixture M2 by

a) separating off propene and, if appropriate, components O>9, HS and HO either completely or partly from the stream II (step IIa) and

b) adding such amounts of components O4-O9, a maximum of 1 mol % of component E and, if appropriate, other saturated or olefinically unsaturated hydrocarbons, based on the amount of components O4-O9 added (step IIb).

The components removed in step IIa are only those components which are finally removed from the system and are thus no longer used for preparing mixture M1, but not those which are initially separated from the mixture M1 or M2 or constituents removed therefrom in an intermediate step or intermediate stage of step I or II but are subsequently recirculated again. This applies analogously to the components added in step IIb, i.e. these include only those which are added to the system from the outside. They consequently do not include components which are initially separated off from the mixture M1 or M2 or a constituent removed therefrom and are subsequently reused for preparing the mixture M1.

An amount of a certain component is also not counted as removed in the sense of step IIa if it is removed from the system but not recirculated but the same amount of the same component originating from another source is once again added to the mixture M1 or M2. This also applies analogously to the components added in IIb.

Steps I and II represent the two steps of a sequence which is repeated a plurality of times, preferably in a continuously operated process in which a permanent stream of circulated material is subjected alternately to steps I and II.

In general, step II is carried out so that the mass of the sum of all materials added corresponds essentially to the mass of the sum of all materials removed.

The removal of the propene from the mixture M2 is preferably carried out by distilling off propene and any ethylene present from the mixture M2 to leave the higher-boiling components and using the ethylene and the distillation residue for preparing the mixture M1. The ethylene which is recirculated in this way is, as explained above, not ethylene which has been included in the calculation of the maximum permitted amount of ethylene added in step IIb.

The removal of components O>9 is likewise preferably carried out by distillation. This is preferably done to prevent accumulation of these components to levels above the defined maximum content in mixture M1.

The addition of the components O4-9 is carried out by adding a mixture M3 comprising components O4-O9, not more than 1 mol % of component E, based on the amount of components O4-O9 added, and, if appropriate, other saturated or olefinically unsaturated hydrocarbons to a mixture M2 from which propene and, if appropriate, component O>9 has been removed in step IIb.

The mixture M3 preferably consists essentially of n-butenes, n-pentenes or n-hexenes or mixtures thereof.

Mixtures produced by FCC cracking or related processes and also n-pentene fractions or hexane fractions isolated from prior metathesis processes can also be used as mixtures M3.

Further possible mixtures M3 are: olefin fractions from thermal cracking processes such as steam cracking or refinery processes (visbreaking, flexicoking, delayed coking). Thermal cracking can be carried out using any feedstocks, for example ethane, propane, butane, raffinate II, naphtha, gas oil or vacuum gas oil.

It is also possible to use mixtures M3 obtained by catalytic cracking (FCC=fluid catalytic cracking or else hydrocracking) in the process of the present invention.

After appropriate work-up, all these streams are suitable as mixtures M3.

The mixture M3 is preferably prepared by

subjecting naphtha or other hydrocarbon compounds to a streamcracking or FCC process and taking off a C₄-hydrocarbon fraction from the stream formed,

preparing a C₄-hydrocarbon stream consisting essentially of isobutene, 1-butene, 2-butene and butanes (raffinate I) from the C₄-hydrocarbon fraction by hydrogenating the butadienes and butynes to butenes or butanes by means of selective hydrogenation or removing the butadienes and butynes by extractive distillation,

separating off the significant proportion of isobutene from the raffinate I by chemical, physicochemical or physical methods to give a raffinate II, and

freeing the raffinate II of catalyst poisons by treatment with adsorber materials to give a mixture M3.

Details of the way in which these steps are carried out are generally known and can likewise be found in DE-A-10013253.

The process of the present invention has the advantage that propene can be prepared from C₄— and higher olefinic hydrocarbons by metathesis using only a small amount, if any, of ethylene introduced from outside the process. This is achieved particularly effectively when the C₄— and higher olefinic hydrocarbons are subjected to an isomerization to form 1- and 2-olefins and ethylene either before or simultaneously with the metathesis. Ethylene and 1-olefins formed in the isomerizing metathesis react with 2-olefins to form propene.

The preferred embodiment of the invention is thus based on the advantageous combination of 4 reactions:

a) self-metathesis of two 1-olefins to form ethylene and an internal olefin (e.g. 1-butene+1-butene→ethylene+3-hexene)

b) ethenolysis of 2-olefins by means of ethylene to form propylene and a 1-olefin (e.g. 2-butene+ethylene→propene+propene or 2-hexene+ethylene→propene+1-pentene)

c) cross-metathesis of 1-olefins and 2-olefins (e.g. 1-butene+2-butene→propene+2-pentene

d) random isomerization to generate a certain proportion of 1- and 2-olefins again (e.g. 1- or 2-hexene from 3-hexene)

1. A process for preparing propene by carrying out the following sequence of steps a plurality of times, wherein
I in step I, a mixture M1 consisting essentially of
unbranched C₄-C₉-monoolefins (components O4-9),
from 0 to 15 mol % of ethylene (component E), based on the components O4-9,
from 0 to 100 mol % of saturated hydrocarbons (components HS), based on the components O4-9,
from 0 to 30 mol %, based on the components O4-9, of olefins having more than 9 carbon atoms (components O>9)
from 0 to 1 mol %, based on the components O4-9, of propene (component O3)
from 0 to 10 mol %, based on the components O4-9, of other hydrocarbons (components HO)
is brought into contact with a metathesis catalyst at from 20 to 350° C., with the proviso that the mole fraction of all 1-olefins and of all 2-olefins as a proportion of the total components O4-9 and O>9 is in each case at least 0.5% if the mixture M1 is not subjected simultaneously or beforehand to an isomerization by means of which the proportion of 1- and 2-olefin is set accordingly, and a mixture M2 is thus prepared,
II in step II, a mixture M1 is prepared from the mixture M2 by
a) removing propene and, if appropriate, components O>9 and components HO either completely or partly and
b) adding components O4-O9, a maximum of 1 mol % of component E, based on the amounts of components O4-O9 added, and, if appropriate, other saturated or olefinically unsaturated hydrocarbons.

2. A process as claimed in claim 1, wherein the metathesis catalyst used is a compound of a metal of transition group VIb, VIIb, or VIII.

3. A process as claimed in claim 1, wherein the isomerization carried out simultaneously with the metathesis in step I is effected either by
using Re₂O₇ on Al₂O₃ as metathesis catalyst and bringing the mixture M1 into contact with the catalyst at from 110 to 350° C. and pressures of from 1 to 60 bar, or
bringing the mixture M1 into contact with catalyst packing which comprises both a metal of transition group VI.b, VII.b or VIII and customary isomerization catalysts.

4. A process as claimed in claim 1, wherein, in step II, the component O>9 is separated off in such amounts that its proportion in M1 does not increase in 2 successive sequences of the steps.

5. A process as claimed in claim 1, wherein the addition of the components O4-9 is effected using a mixture M3 consisting essentially of n-butenes, n-pentenes or n-hexenes or mixtures thereof.

6. A process as claimed in claim 5, wherein the mixture M3 is prepared by
subjecting naphtha or other hydrocarbon compounds to a streamcracking or FCC process and taking off a C₄-hydrocarbon fraction from the stream formed,
preparing a C₄-hydrocarbon stream consisting essentially of isobutene, 1-butene, 2-butene and butanes (raffinate I) from the C₄-hydrocarbon fraction by hydrogenating the butadienes and butynes to butenes or butanes by means of selective hydrogenation or removing the butadienes and butynes by extractive distillation,
separating off the significant proportion of isobutene from the raffinate I by chemical, physicochemical or physical methods to give a raffinate II, and
freeing the raffinate II of catalyst poisons by treatment with adsorber materials to give a mixture M3.

7. A process as claimed in claim 1, wherein the propene is separated off from the mixture M2 by distilling off propene and ethylene from the mixture M2 to leave the higher-boiling components and using the ethylene and the distillation residue for preparing the mixture M1.

8. A process as claimed in claim 1, wherein the multiple repetition of the sequence of steps I and II is carried out as a continuously operated process having a permanent stream of circulated material which is alternately subjected to the steps I and II.
